(19) United States
(12) Patent Application Publication
(10) Pub. No.: US 2026/0108652 A1
(43) Pub. Date: Apr. 23, 2026

(54) DEVICE FOR DISPENSING VOLATILE SUBSTANCES, IN PARTICULAR FRAGRANCES AND/OR ACTIVE SUBSTANCES

(71) Applicant: CTR, LDA, Samora Correia (PT)

(72) Inventors: Harold AUGIER, Zhongshan City (CN); Ren JIANHUI, Zhongshan City (CN); Wei HONG, Zhongshan City (CN)

(21) Appl. No.: 19/115,373

(22) PCT Filed: Sep. 26, 2022
(86) PCT No.: PCT/EP2022/076647

§ 371 Date: Mar. 26, 2025

(51) Int. Cl. A61L 9/03

(52) U.S. Cl. CPC A61L 9/037; A61L 2209/134

(57) ABSTRACT

A device for dispensing, in particular for vaporizing, volatile substances, in particular fragrances and/or active substances, has a housing with an outflow opening for a substance hot-air flow generated in the housing, a flow guiding device provided in the region of the outflow opening, which is configured to direct the substance hot-air flow flowing out of the outflow opening in a predetermined direction and to generate a directed plume. The device preferably has an electrical heating device with a heating element for generating the substance hot-air flow. The flow guiding device for directional deflection of the plume has a deflection surface pointing in the direction of a deflection in the region of the outflow opening, preferably at a distance therefrom, by way of which the plume can be deflected, in particular by a Coanda effect.

The invention refers to a device for dispensing, in particular for vaporizing, volatile substances, in particular fragrances and/or active substances, according to the preamble of claim 1, and to a method according to the preamble of claim 17.

Such devices for dispensing, in particular for vaporizing, volatile substances, in particular fragrances and/or active substances, are generally known in various designs and regularly comprise a housing with at least one outflow opening for a substance hot air flow generated in the housing as a plume. A container with the substance to be dispensed is inserted into the housing. The container has a wick which protrudes from the container with a wick end forming a substance delivery region in the region of the at least one outflow opening. The device also has an electrical heating device with an electrical heating element arranged in the region of the wick end. Thus, when the heating device is activated, heat is applied to the free wick end in order to be able to quickly release the substance accumulating in the free wick end to the environment or to evaporate it. Such a structure is known, for example, from WO 98/58692 A1. In order to be able to variably adjust the degree of evaporation and thus the evaporation output, the container together with the wick is mounted in a height-adjustable manner in the housing of the device, whereby the relative position of the wick to the heating device can be changed and the heating output of the heating device fixed to the housing remains unchangeably the same.

In a generally known manner, such devices are designed as plug-in components, which have plug-in contacts protruding from the housing, which in turn can be plugged into a socket on the room wall side for supplying the heating device with electrical energy, with the device thereby being held on a room wall at the same time. Thus, in its functional position, the device is arranged very close to a room wall, which has the disadvantage that the generated plume is produced close to the room wall and essentially flows upwards along the latter after it has been emitted, so that the desired substance effect only sets in as a result of subsequent turbulence and distribution in the room. Particularly after initial activation of the device at a still low substance release rate, the plume can partially condense in the insertion area of the device and thus lead to unsightly discolorations and stains on the wall in this area.

This problem can be reduced by deflecting the plume away from a room wall at an angle upwards into the room after the plume discharge opening. To this end, it is known (WO 2017/215726 A1) to generate, in addition to the plume in the housing, a further substance-free hot air flow which is fed obliquely to the plume with the aid of a flow guide element and, after mixing, is directed obliquely upwards away from a room wall as a plume hot air flow into a room for further propagation.

In a known similar device for dispensing, in particular for evaporating, volatile substances (US 2020/0171190 A1), a generated plume is likewise deflected upwards away from a room wall into a room, in which a lip-like formation pointing obliquely upwards in the direction of deflection is attached directly to the edge of a plume outlet opening, by means of which a plume deflection can be carried out, in particular by utilizing a Coanda effect. This arrangement causes disadvantageous problems at such a lip by reducing the plume velocity in connection with condensations.

It is therefore an object of the invention to propose a relatively simple and inexpensive device with which, after activation and/or if necessary, a rapid, condensation-free distribution of the plume with the substances contained can be achieved starting from the device upwards into a room and away from a nearby room wall.

This object is solved with the features of the independent patent claims. Advantageous embodiments are the subject of the related subclaims.

According to claim 1, a device for dispensing, in particular for vaporizing, volatile substances, in particular fragrances and/or active substances, is provided which has a housing which has at least one outflow opening for a substance hot-air flow generated in the housing. In the region of the at least one outflow opening, at least one flow guiding device is provided which is suitable and designed to guide the substance hot air flow flowing out of the at least one outflow opening in a predetermined direction and to generate a directed plume. Optionally, it may be provided that the device comprises an electrical heating device comprising at least one heating element for generating the substance hot air flow. According to the invention, it is provided that the flow guiding device for directing the direction of the plume has a deflection surface facing or pointing in the direction of a deflection in the region of the outflow opening, preferably at a distance therefrom, through which the plume can be deflected, in particular by a Coanda effect.

Such a design, in conjunction with the directional control of the plume, advantageously helps to avoid undesirable condensation and vortex formation as well as a reduction in the discharge velocity, as will be shown below.

Such a concrete structure with a deflection surface in the area of the outflow opening can in principle be used in conjunction with any device which serves to dispense substances. However, it is particularly preferred to use it in conjunction with a structure in which the device has a container which is connectable to the housing, preferably insertable into the housing, and which has the substance to be dispensed. The container in turn preferably has a wick which protrudes from the container with a wick end forming a substance dispensing area in the region of the at least one outflow opening. Such a structure is simple and inexpensive to manufacture and is also characterized by a high degree of functional reliability. Moreover, the term wick is to be understood here expressly in a broad and comprehensive sense and expressly includes any conveying device with which the substance can be conveyed, in particular any capillary element which is suitable and designed to transport and convey substances by means of capillary effect. In this respect, as is customary in the present field, the term wick is merely representative of a large number of possible embodiments of conveying devices or capillary elements made of any suitable material.

As the inventor's tests have shown, undesirable condensation and vortex formation as well as a reduction in the outflow velocity can be avoided in particular if the outflow opening is arranged, preferably approximately centrally, on an upper side of the housing having and/or forming the flow guiding device, preferably on a cover shell of the housing having and/or forming the flow guiding device. Particularly in connection with a structure comprising a container with wick as described above, it is furthermore advantageous if the upper side, preferably the cover shell, of the housing is located in this case above the heating device and/or the outflow opening is located above the wick end, as seen in the vertical axis direction.

In addition, it has proved particularly advantageous that an upper side wall of the upper side, preferably of the upper shell, which surrounds the edge at least in regions resp. certain areas and opens upwards in the vertical axis direction and/or outwards in the radial direction, is offset at a radial distance from the outflow opening, preferably offset from the approximately centrally located outflow opening. This is because an almost plume-free free space is formed in the immediate vicinity of the outflow opening, which means that the plume flow is not slowed down and no undesirable condensation occurs in the outflow area.

A particularly advantageous directional deflection of the plume in the desired direction can also be achieved by the height of the top wall, seen radially outwards, rising from a base region, preferably steadily, to a radially opposite region of greater height, the region of greater height forming a deflection region with the deflection surface, at which the plume can be deflected with respect to a vertical direction in an outflow direction running resp. extending obliquely upwards, over the deflection region.

For a targeted outflow and discharge of the plume, which helps to avoid vortex and condensate formation, it has also proved advantageous if the outflow opening is formed in a crater-like bulge of the upper side, which is preferably formed by a bottom wall of the cover shell. According to a particularly preferred concrete embodiment, the diameter of the bulge is small compared to the diameter of the upper side, preferably of the cover shell, and/or the height of the bulge is small in relation to the wall height of the deflection surface.

A structure in which the transitions between the housing wall forming the deflection surface and the upper side, preferably the cover shell, and/or between the upper side, preferably the cover shell, and the bulge are rounded can further contribute to the avoidance of vortex formation at tear-off edges. The same applies analogously to another preferred embodiment according to which the housing wall edge forming an upper free end of the deflection surface, viewed in the vertical axis direction, and/or the edge of the crater-side outflow opening are rounded.

The above concrete measures and designs, in conjunction with the directional control of the plume, thus all contribute to preventing undesired condensation, vortex formation and reduction of the outflow velocity, whereby, as described above, the measures can also be implemented individually or in alternating combinations with one another, depending on the application, and do not all have to be implemented simultaneously, although the latter is of course fundamentally advantageous.

When we speak above of a cover shell, we are referring here to a component forming the upper side of the housing, which can be a separate housing component, e.g. as a separate shell part placed on the housing, or of course can also be formed and/or shaped as a single piece of material with the housing. In the functional position or viewed in the vertical axis direction, the upper side or cover shell is preferably aligned approximately horizontally or extends in a horizontal plane.

It is also particularly advantageous if the deflection surface is arranged on the housing in such a way that, in the functional position of the device, the deflection surface is directed away from an area to which no flow is to be directed, preferably away from a room wall, and in particular, in the case of a device designed as a plug-in component, is located and arranged opposite the plug-in contacts. This prevents plume flows on a room wall, which can result in unsightly discoloration of the building wall.

Particularly advantageous is further a structure in which the device has an electrical heating device which has at least one heating element for generating the substance hot air flow. Preferably, the electrical heating device has a control device which is suitable and designed for setting different heating powers.

In a more specific embodiment, the electrical heating device is an electrical resistance heater with a heat-generating overall resistance that can be activated for heat generation, wherein the electrical heating device can be connected to an energy source for energy supply and can be supplied with a predetermined electrical voltage, wherein it is preferably provided that the heat-generating overall resistance is formed by the at least one heating element. Such a structure is characterized by a high degree of functional reliability and is also easy to manufacture.

In addition, a structure in which the control device is suitable and designed to change the overall heat-generating resistance of the heating device in order to set the heating power of the heating device is particularly advantageous. Alternatively or additionally, the control device can be suitable and designed to change the electrical voltage in order to adjust the heating power of the heating device.

For reliable operation of the device, it is also advantageous if the at least one heating element comprises a heating body made of a thermally conductive material and at least one electrical resistor element thermally coupled to the heating body, preferably at least partially integrated in the heating body. A single heating body may also include two or optionally more resistor elements to form a plurality of interconnected heating elements.

In a particularly preferred embodiment, the device can be designed as a plug-in component and have plug-in contacts projecting from the housing, which are suitable and designed to be plugged into a socket, preferably on the room wall side, for supplying the heating device with electrical energy and for holding the device.

In its functional position, the device, preferably as a plug-in component in the plugged-in state in a socket, preferably on the room wall side, is to be aligned with its housing in such a way that the wick associated with the heating device, preferably the wick surrounded by the heating device, stands approximately vertically in the housing and the outflow opening in the housing is located at a distance above it.

Furthermore, the object is solved with respect to the method for dispensing, in particular for vaporizing, volatile substances, in particular fragrances and/or active substances, with the features of claim 17. The advantages resulting from the method according to the invention correspond analogously to those of the device according to the invention, so that reference is made to the explanations previously given in order to avoid repetition.

The advantageous embodiments and further developments of the invention explained above and/or reproduced in the subclaims can be used individually or also in any combination with one another-except, for example, in the cases of clear dependencies or incompatible alternatives.

The invention and its advantageous embodiments and further developments are explained in more detail below with reference only to exemplary and schematic drawings.

The drawings show:

FIG. 1 a perspective view of an exemplary embodiment according to the invention of a device for dispensing, in particular for vaporizing, volatile substances, in particular fragrances and/or active substances, with a housing, an inserted container and plug contacts for connection to a socket,

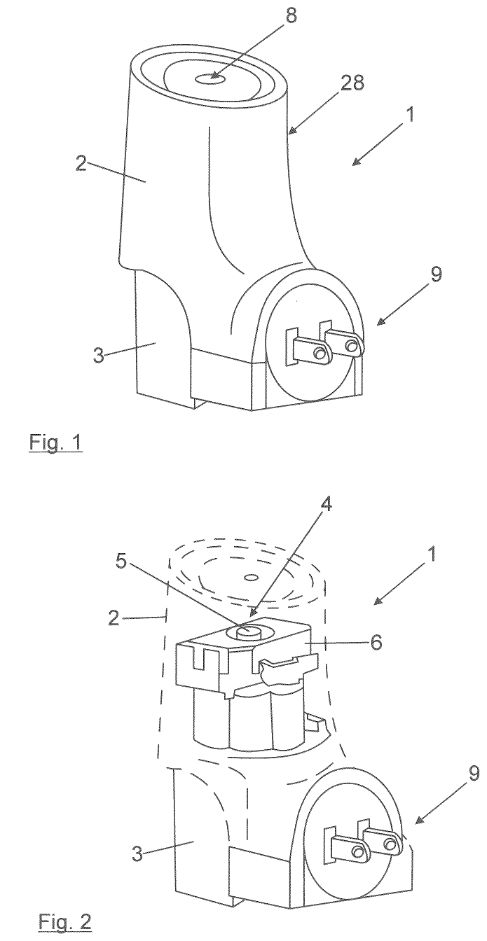

FIG. 2 a view corresponding to FIG. 1, without the housing, which is only indicated by dashed lines, with the internal structure with a heating device and a wick,

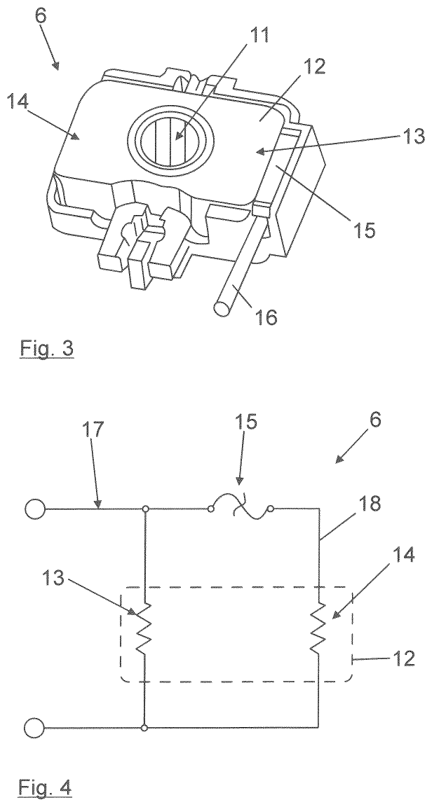

FIG. 3 a top perspective view of the heating device,

FIG. 4 a schematic circuit diagram of a first embodiment with two electrical heating elements connected in parallel and a thermal switch formed by an R.TCO,

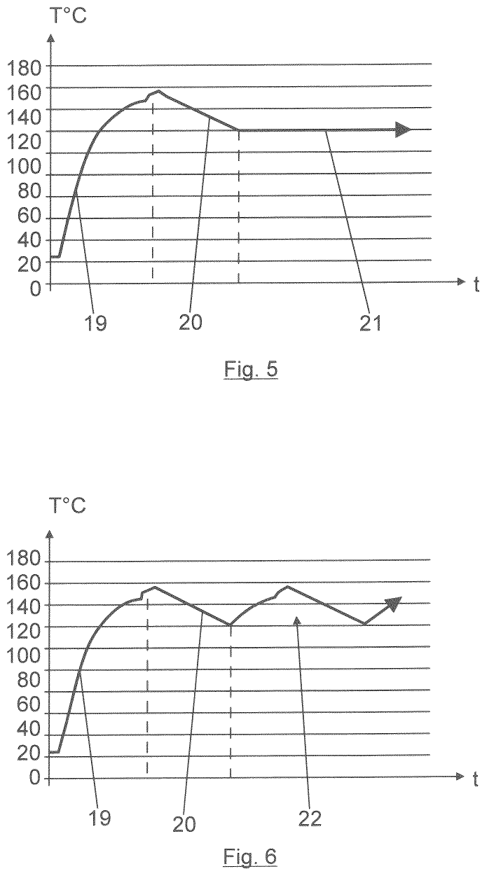

FIG. 5 a view corresponding to FIG. 1 with a drawn section line of a vertical longitudinal section through the device,

FIG. 6 a schematic sectional view of the longitudinal section of FIG. 5 in the upper region of the device with schematic representation of a deflected outflowing plume, and

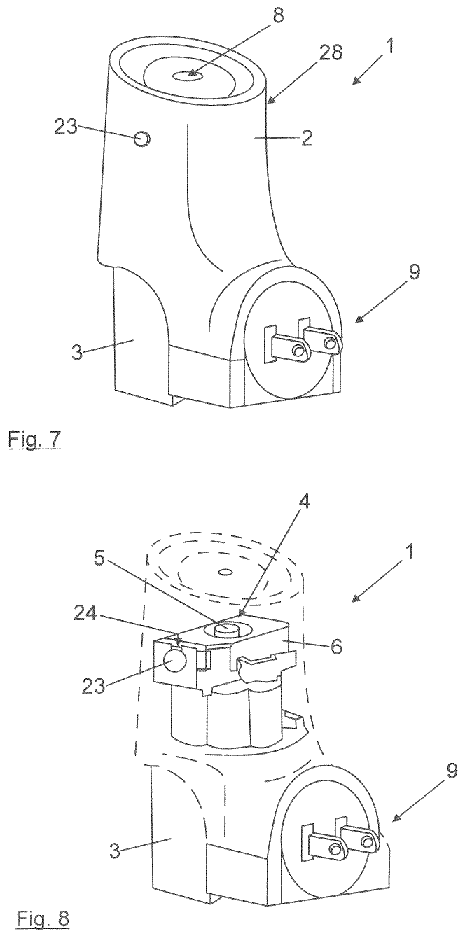

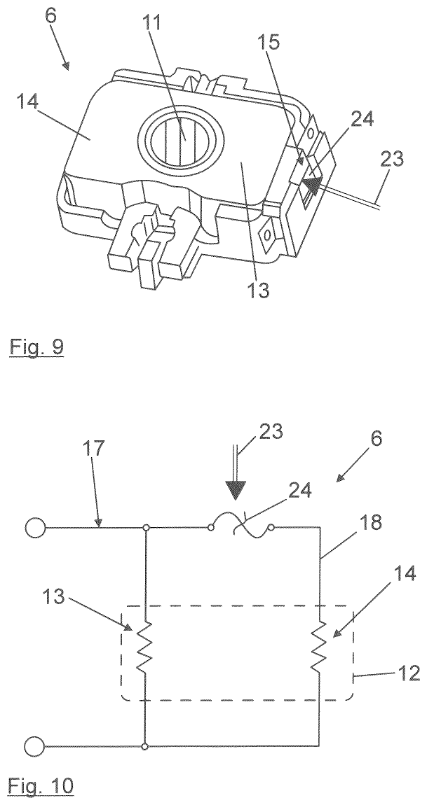

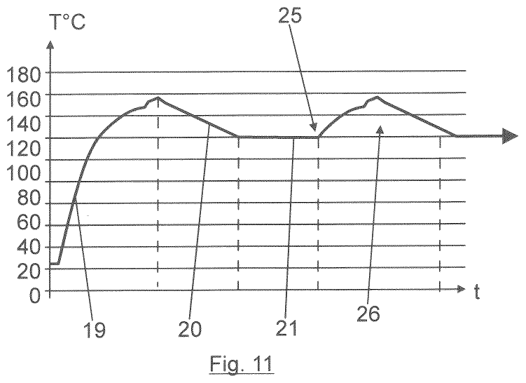

FIG. 7 a schematic representation of a device mounted in a room on the wall side with plume flowing out and spreading in the room.

FIG. 1 shows a device 1 for vaporizing volatile substances, in particular fragrances and/or active substances, with a housing 2 in which a container 3 containing the substance to be dispensed is inserted. From the internal structure of the device 1 shown in FIG. 2, it is apparent that a wick 4 received in the container protrudes from the container with a wick end 5 through a heating device 6. After activation of the heating device 6, heat is transferred to the wick end and thus a substance hot air flow is generated which forms a plume 7 (see FIGS. 6 and 7). An outlet opening 8 is provided in the housing 2 above the wick end 5.

The device 1 is, merely exemplary here, L-shaped in its basic design and forms a plug-in component. In its functional position, the housing 2 is aligned in such a way that the wick 4 surrounded by the heating device 6 is approximately vertical in the housing 2, namely with the outflow opening 8 located at a distance above it.

On a lower L-leg of the housing 2, plug contacts 9 protrude from the housing 2, which can be plugged into a socket 10 on the room wall side (see FIG. 7) for supplying the heating device 6 with electrical energy and for holding the device 1 on the associated room wall.

FIG. 3 shows an electric heater 6 with a central wick-receiving opening 11 in a heater body 12. The heater body 12 consists of a thermally conductive material in which two electrical heating elements 13, 14 with (not visible) integrated electric resistor elements are contained. FIG. 3 also schematically shows a thermoswitch 15 and an electric cable 16 for the electric power supply.

The electrical circuit in connection with the heating function is shown in FIG. 4:

From the schematic circuit diagram of FIG. 4 it can be seen that the heating element 13 and the heating element 14 are connected in parallel in an electrical heating circuit 17, whereby, just for example, after activation of the heating circuit 17 the heating element 13 is permanently switched on and the heating element 14 can be, according to its temperature rise, switched off and, if necessary, switched on again with the thermoswitch 15 in its supply line 18.

In FIG. 5, the device 1 is shown in connection with a section line A-A for a vertical longitudinal section between the two connector elements 9. The upper area of this section is shown schematically in FIG. 6.

A flow guiding device 27 is formed in the upper region of the device 1, in which the outflow opening 8 is arranged in a crater-like manner and here merely exemplarily approximately centrally at the bottom of a cover shell 28 of the housing 2, which is here likewise merely exemplarily concave. The cover shell 28 lies at a distance above and over the heating device 6 and the outflow opening 8 also lies above and over the wick end 5, as can be seen from FIG. 2. An upper side wall or shell side wall 29 of the upper shell 28, which surrounds the edge at least in some areas, preferably completely, and opens upwards in the vertical axis direction and/or outwards in the radial direction, runs offset at a radial distance 30 from the outflow opening 8, which is arranged approximately centrally here.

The height of the shell sidewall 29 increases steadily from a base region or region 38 of lesser height to a radially opposite region 31 of greater height. The region 31 of greater height, together with a deflection surface 32, forms the deflection region 31 at which the plume 7 flows away from a nearby room wall in an oblique outflow direction 34 extending upwardly across the deflection region 31 relative to a perpendicular direction 33.

The deflection takes place in particular by means of a Coanda effect, whereby an almost plume-free free space 36 is formed by the distance 30 between the outflow opening 8 and the deflection area 31, so that, in particular also supported by the roundings, the plume flow is not slowed down and no undesirable condensation occurs at the outflow area.

FIG. 7 shows the activated device 1 schematically in its functional position. It is plugged into a socket 10 in the lower area of a room wall 35 for power supply and initial activation as well as for a housing holder. As shown in FIG. 6, the plume 7 extends obliquely upward from the device 1 at a deflection angle away from the room wall 35 in order to dissolve in a swirl zone 37 after an approximately laminar jet area and to distribute itself in the room.

List of reference signs

| | |
|---|---|
| 1 | Device |
| 2 | Housing |
| 3 | Container |
| 4 | Wick |
| 5 | Wick end |
| 6 | Heating device |
| 7 | Substance hot air flow (plume) |
| 8 | Outflow opening |
| 9 | Plug contacts |
| 10 | Socket |
| 11 | Wick-receiving opening |
| 12 | Heater body |
| 13 | Heating element (high PTC) |
| 14 | Heating element (low PTC) |
| 15 | Thermoswitch (R. TCO) |
| 16 | Electrical cables |
| 17 | Heating circuit |
| 18 | Cable |
| 27 | Flow guiding device |
| 28 | Cover shell |
| 29 | Shell sidewall |
| 30 | Distance |
| 31 | Deflection range |
| 32 | Deflection surface |
| 33 | Perpendicular direction |
| 34 | Outflow direction |
| 35 | Room wall |
| 36 | Open space |
| 37 | Swirl zone |
| 38 | Area |

1-17. (canceled)

18. A device for dispensing volatile substances, the device comprising:
a housing formed with at least one outflow opening for a substance hot-air flow generated in said housing;
at least one flow guiding device disposed in a region of said at least one outflow opening, said flow guiding device being configured to guide the substance hot-air flow flowing out of said at least one outflow opening in a predetermined direction and to generate a directed plume;
said flow guiding device having a deflection surface pointing in a direction of a deflection in the region of said at least one outflow opening, for directionally guiding the plume and deflecting the plume by a Coanda effect.

19. The device according to claim 18, comprising:
a container to be connected to said housing and containing the substance to be dispensed;
said container having a wick projecting out of said container and having a wick end forming a substance dispensing area in the region of said at least one outflow opening.

20. The device according to claim 18, wherein said outflow opening is formed on an upper side of said housing having said flow guiding device.

21. The device according to claim 20, wherein said outflow opening is formed approximately centrally on a cover shell of said housing that is formed with said flow guiding device.

22. The device according to claim 20, which comprises a cover shell forming the upper side of said housing located above said heating device and/or wherein said outflow opening is located above a wick end of a wick in a vertical axis direction.

23. The device according to claim 20, wherein an upper side wall of said upper side which surrounds an edge at least in regions and opens upwards in a vertical axis direction and/or outwards in a radial direction, runs at a radial distance offset from said outflow opening.

24. The device according to claim 23, wherein a height of said upper side wall, seen radially outwards, rises from a base region to a radially opposite region of greater height, the region of greater height forming a deflection region with said deflection surface, at which the plume is deflected with respect to a vertical direction in an outflow direction running obliquely upwards over said deflection region.

25. The device according to claim 20, wherein said outflow opening is formed in a crater-shaped bulge of said upper side, being a bottom wall of said cover shell.

26. The device according to claim 25, wherein a diameter of said bulge is small compared to a diameter of the top surface, and at least one of said cover shell or a height of said bulge is small in relation to a wall height of said deflection surface.

27. The device according to claim 20, wherein transitions between said housing wall forming said deflection surface and the upper side of said housing and/or transitions between the upper side and said bulge are rounded.

28. The device according to claim 20, wherein transitions between said housing wall forming said deflection surface and said cover shell and/or transitions between said cover shell and said bulge are rounded.

29. The device according to claim 18, wherein at least one of a housing wall edge forming an upper free end of said deflection surface, viewed in a vertical axis direction, or an edge of a crater-side outflow opening are rounded.

30. The device according to claim 18, wherein said deflection surface is formed on said housing such that, in a functional position of the device, said deflection surface is directed away from an area to which no flow is to be directed.

31. The device according to claim 30, wherein the device is a plug-in component and said deflection surface is located opposite plug-in contacts of the device and configured to guide the plume away from a room wall.

32. The device according to claim 18, which comprises an electrical heating device having at least one heating element for generating the substance hot air flow, and said electrical heating device includes a controller configured for setting different heating powers.

33. The device according to claim 32, wherein said electrical heating device is an electrical resistance heater with a heat-generating total resistance that is activatable for heat generation, wherein said electrical heating device is connectable to an energy source for energy supply and is to be supplied with a predetermined electrical voltage, and wherein the heat-generating total resistance is formed by said at least one heating element.

34. The device according to claim 32, wherein said at least one heating element comprises a heating body made of a thermally conductive material and at least one electrical resistor element thermally coupled to said heating body.

35. The device according to claim 32, configured as a plug-in component having plug-in contacts which project out of said housing and are configured to be plugged into a socket on a room wall for supplying said heating device with electrical energy and for holding the device.

36. The device according to claim 18, wherein the device in a functional position as a plug-in component in a plugged-in state in a socket, is aligned with said housing and a wick associated with said heating device, and stands approximately vertically in said housing and said outflow opening in said housing is located at a distance above said wick.

37. A method, comprising:
providing the device according to claim 18; and
vaporizing and dispensing volatile substances being one or fragrances or active substances with the device.
